(19) United States
(12) Patent Application Publication
(10) Pub. No.: US 2015/0165128 A1
(43) Pub. Date: Jun. 18, 2015

(54) SYRINGE

(71) Applicant: SANG-A FRONTEC CO., LTD., Incheon (KR)

(72) Inventors: WONHYUN SEOK, INCHEON (KR); JOON HWAN CHUN, INCHEON (KR); SOO-MIN CHAE, INCHEON (KR)

(73) Assignee: SANG-A FRONTEC CO., LTD., Incheon (KR)

(21) Appl. No.: 14/108,396

(22) Filed: Dec. 17, 2013

(51) Int. Cl. A61M 5/31

(52) U.S. Cl. CPC A61M 5/3145

(57) ABSTRACT

Injection liquid is inhaled into a cylinder of a syringe through an injection needle, a passing hole and an inhalation path in order. The liquid in the syringe is injected into a patient's body through a discharge path, a filter and the passing hole in order. The injection occurs a filter holder is rotated at the same time with a movement of a pressure rib along a spiral groove when a needle holder carries out a rectilinear motion.

### BACKGROUND OF THE INVENTION

1. Field of the Invention

The present invention relates to a syringe.

More particularly, the present invention relates to a syringe which smoothly inhales injection liquid without going through a filter when inhaling the injection liquid into the syringe and filters foreign matters contained in the injection liquid by making the injection liquid go through the filter when injecting the inhaled injection liquid into a human body.

2. Background Art

In general, a syringe includes: a cylinder having a space for storing injection liquid therein; a piston moving back and forth inside a chamber formed in the cylinder in order to inhale or discharge the injection liquid; a needle holder forcedly fit to an outer circumferential surface of a neck part formed at the front of the cylinder; and an injection needle joined to the tip of the needle holder.

When a user breaks an upper part of an ampoule which contains injection liquid therein in order to open the ampoule and retreats the piston after putting the injection needle into the opened part, the injection liquid contained in the ampoule is inhaled into the cylinder. In the above-mentioned state, when the user sticks the injection needle into a patient's affected part and moves the piston forward, the injection liquid contained inside the cylinder is injected into the patient through the injection needle.

However, when the user breaks the upper part of the ampoule in order to inhale and put the injection liquid, there is a risk that pieces of broken glass may get into the ampoule, and because the pieces of broken glass are put into the syringe when the syringe inhales the injection liquid, it has a fatal influence on the patient when the injection liquid is injected into the human body.

In order to solve the above-mentioned problem, Korean Patent No. 10-981586 (hereinafter, called ‘prior patent’) discloses a syringe which inhales injection liquid after a filter tip having a filter mounted therein is inserted into a neck part of a cylinder so as to previously filter pieces of broken glass contained in the injection liquid.

However, the prior patent has several problems in that the process to inhale and inject the injection liquid is complicated and filter pressure acting by the filter during the process of inhaling the injection liquid hinders a smooth inhalation of the injection liquid because the user has to make the syringe inhale the injection liquid after inserting the filter tip into the neck part of the cylinder in order to inhale the injection liquid but injects the inhaled injection liquid of the syringe into the patient's body after removing the filter tip and joining a new syringe tip to the syringe in order to inject the injection liquid into the patient's body.

Moreover, the prior patent has another problem in that manufacturing costs are increased because it needs a separately manufactured filter tip.

### SUMMARY OF THE INVENTION

Accordingly, the present invention has been made to solve the above-mentioned problems occurring in the prior arts, and it is an object of the present invention to provide a syringe which promotes inhalation of the syringe because there is no filter pressure when the syringe inhales injection liquid and filters foreign matters contained in the injection liquid when the inhaled injection liquid of the syringe is injected into a human body, thereby enhancing inhalation of the syringe and providing a filtering effect.

It is another object of the present invention to provide a syringe which includes a filter block having an inhalation path and a discharge path disposed separately, a pressure rib formed on the inner face of a needle holder to which the filter block is inserted, and a spiral groove formed in the outer face of the filter block in such a way that the pressure rib is joined to the spiral groove such that a route of the injection liquid is changed by a rectilinear motion of the needle block, thereby providing convenience in use because there is no need to replace the filter tip of the conventional syringe.

It is a further object of the present invention to provide a syringe which prevents a separation of the needle block and allows the user to recognize whether or not the piston containing the injection liquid is correctly connected with the discharge path, thereby providing convenience in use.

To accomplish the above object, according to the present invention, there is provided a syringe including: a filter block having an inhalation path and a discharge path such that injection liquid is inhaled into a cylinder through the inhalation path and is injected into a patient's body through the discharge path communicating with the cylinder when the filter block is rotated after the inhalation of the injection liquid; and a filter disposed on the discharge path for filtering the injection liquid when the injection liquid is injected into the patient's body.

Moreover, the present invention provides a syringe for inhaling and discharging injection liquid by a reciprocating motion of a piston of a cylinder, including: a connecting cap joined to the front end of the cylinder, the connecting cap having an inhalation path and a discharge path communicating with the inside of the cylinder; a filter block assembled to the front face of the connecting cap, the filter block having a passing hole selectively communicating with the inhalation path and the discharge path and a spiral groove formed on the outer face of the filter block; a filter fit to the front face of the connecting cap, the filter having a hole formed at the same position as the inhalation path so as to pass the inhaled injection liquid without filtering; a pad located on the front face of the filter and having a hole communicating with the passing hole of the filter block; and a needle holder having an injection needle disposed at the front portion and first and second mounting parts disposed therein, the filter block and the connecting cap being fit to the needle holder in order, the needle holder having a pressure rib disposed in the first mounting part inserted into the spiral groove, wherein the injection liquid is inhaled into the cylinder through the injection needle, the passing hole and the inhalation path in order, and is injected into a patient's body through the discharge path, the filter and the passing hole in order while the filter holder is rotated at the same time with a movement of the pressure rib along the spiral groove when the needle holder carries out a rectilinear motion.

First, the syringe according to the exemplary embodiment of the present invention can smoothly inhale the injection liquid without going through the filter when inhaling the injection liquid and filter foreign matters (pieces of broken glass of the ampoule) contained in the injection liquid by making the injection liquid go through the filter when injecting the inhaled injection liquid into the human body, thereby providing easiness in use and a filtering effect.

Second, the syringe according to the exemplary embodiment of the present invention is convenient in use and stably fixes the needle holder while injecting the injection liquid because the syringe easily converts an inhalation path into a discharge path just by straightly moving the needle holder to which the injection needle is joined.

Third, the syringe according to the exemplary embodiment of the present invention can be used conveniently in safety because it makes the user recognize the state where the syringe is converted into the discharge path in order to inject the injection liquid into the human body.

Fourth, the syringe according to the exemplary embodiment of the present invention smoothly inhales the injection liquid and provides the filtering effect using just one filter member, differently from the conventional syringe requiring the filter tip and the syringe tip, thereby reducing the manufacturing costs.

### BRIEF DESCRIPTION OF THE DRAWINGS

The above and other objects, features and advantages of the present invention will be apparent from the following detailed description of the preferred embodiment of the present invention in conjunction with the accompanying drawings, in which:

FIG. 1 is a perspective view of a syringe according to a preferred embodiment of the present invention;

FIG. 2 is an exploded sectional view of the syringe;

FIG. 3 is an enlarged sectional view of essential parts of the syringe;

FIGS. 4a and 4b are respectively a perspective view and a sectional view of a needle block applied to the syringe according to the preferred embodiment of the present invention;

FIGS. 5a and 5b are respectively a perspective view and a sectional view of a filter block applied to the syringe according to the preferred embodiment of the present invention;

FIG. 6 is an enlarged sectional view of the essential parts of the syringe;

FIGS. 7a and 7b are front views of a filter and an adjustment member applied to the syringe according to the preferred embodiment of the present invention;

FIGS. 8a and 8b are sectional views showing an operation process of the syringe according to the preferred embodiment of the present invention; and

FIGS. 9a and 9b are perspective view of a syringe according to another preferred embodiment of the present invention.

### DETAILED DESCRIPTION OF THE PREFERRED EMBODIMENT

Hereinafter, preferred embodiments of the present invention will be described with reference to the attached drawings as follows. The preferred embodiments of the present invention are described enough to be easily understood by those skilled in the art, but the present invention is not restricted to the preferred embodiments. It will be understood by those of ordinary skill in the art that various changes, modifications and alternations may be made therein without departing from the technical idea and the technical scope of the present invention. In this specification, the term ‘and/or’ has the meaning to include at least one of components listed in the front and rear. In this specification, to locate ‘on’ some component means not only that a component is directly located on another component but also that another component may be located on the component. In this specification, components or parts are called in the name of “first”, “second”, and so on, but are not restricted to the above-mentioned terms because they are terms used for clear explanations. Thickness and relative thickness of components of the present invention provided in the drawings may be exaggerated in order to clearly describe the preferred embodiments of the present invention. Furthermore, matters expressed in the attached drawings are illustrated in order to easily describe the preferred embodiments of the present invention and may be different from the forms embodied actually.

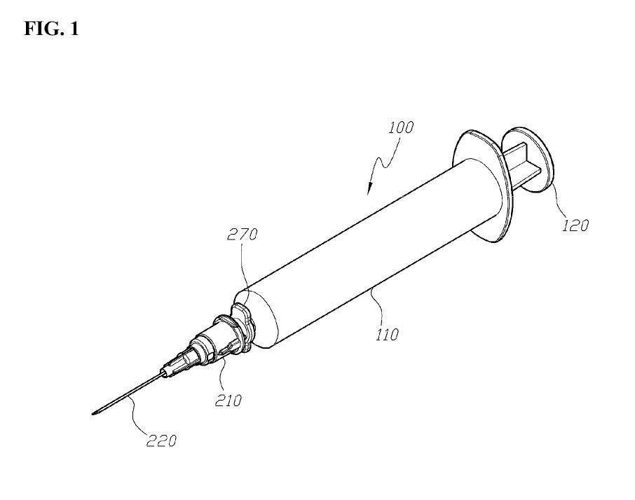

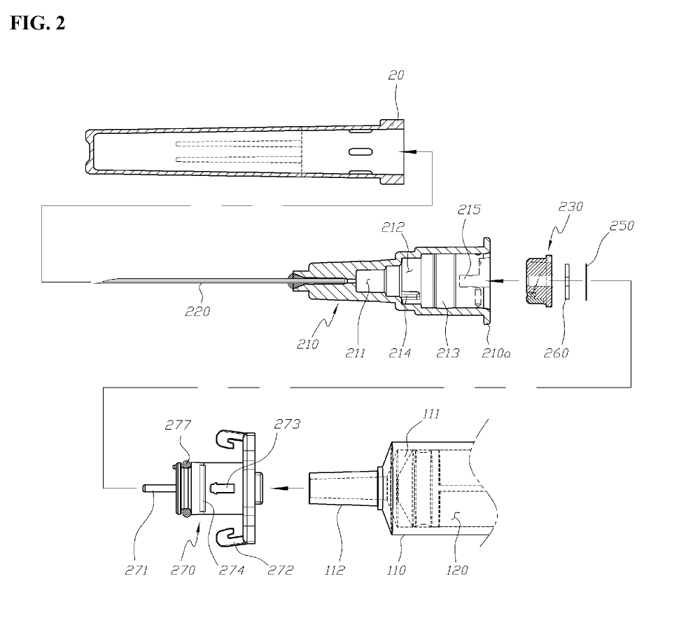

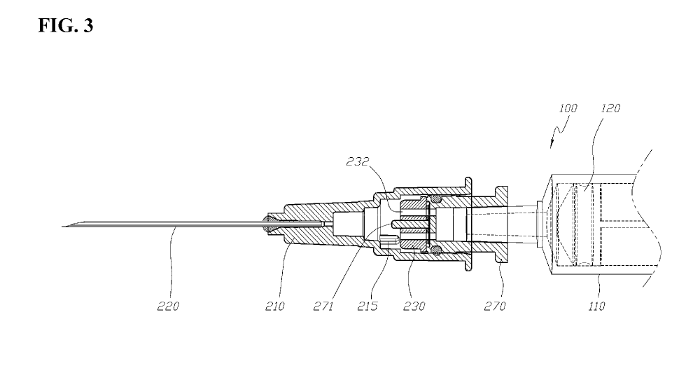

FIG. 1 is a perspective view of a syringe according to a preferred embodiment of the present invention, FIG. 2 is an exploded sectional view of the syringe, and FIG. 3 is an enlarged sectional view of essential parts of the syringe.

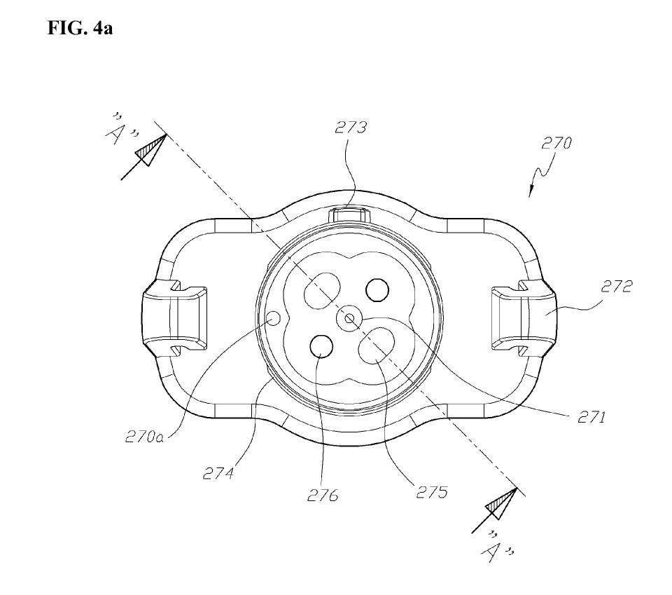

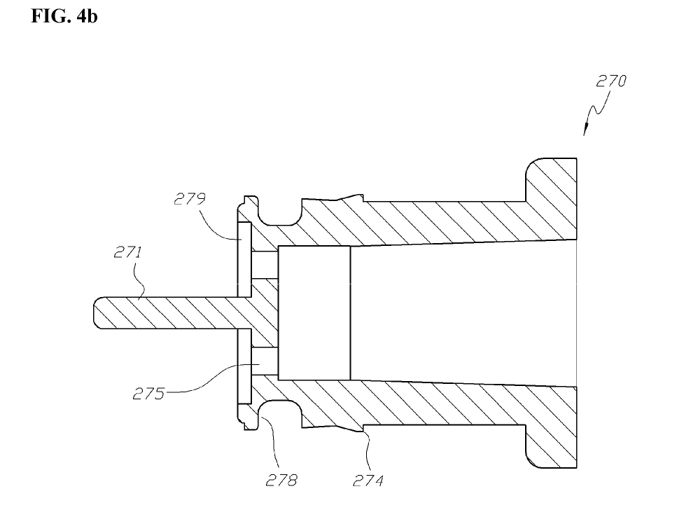

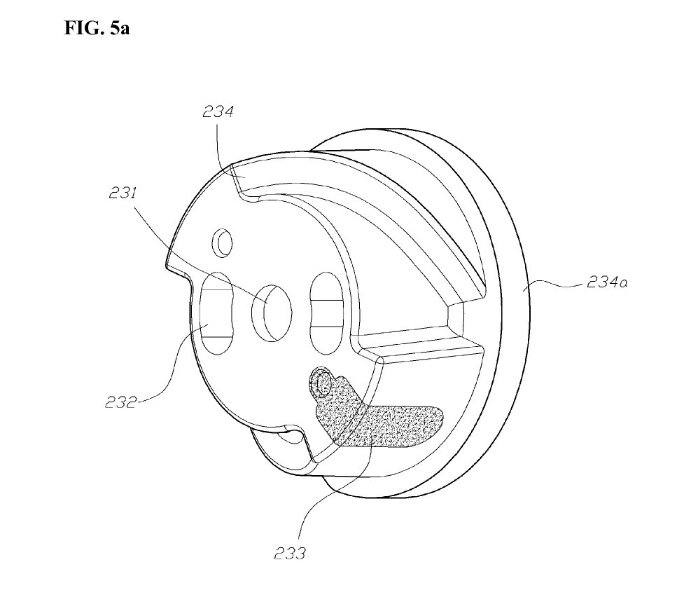

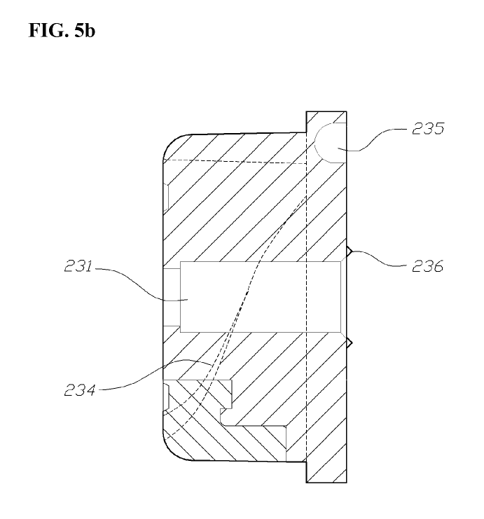

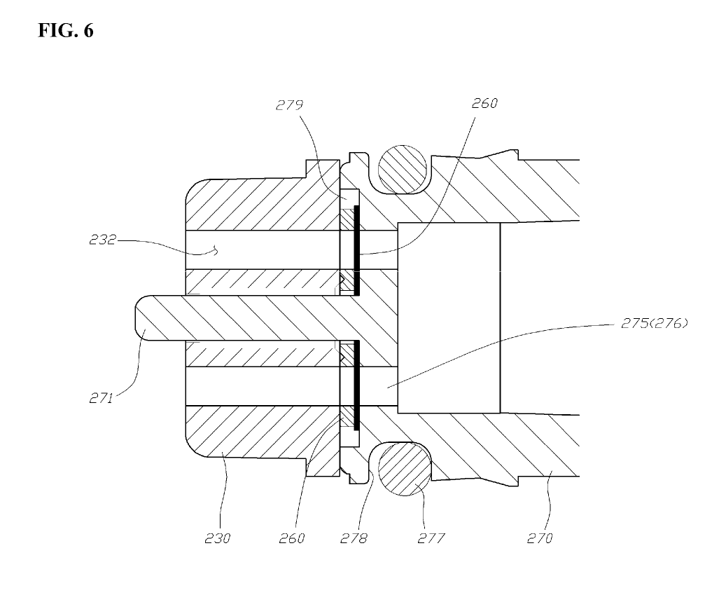

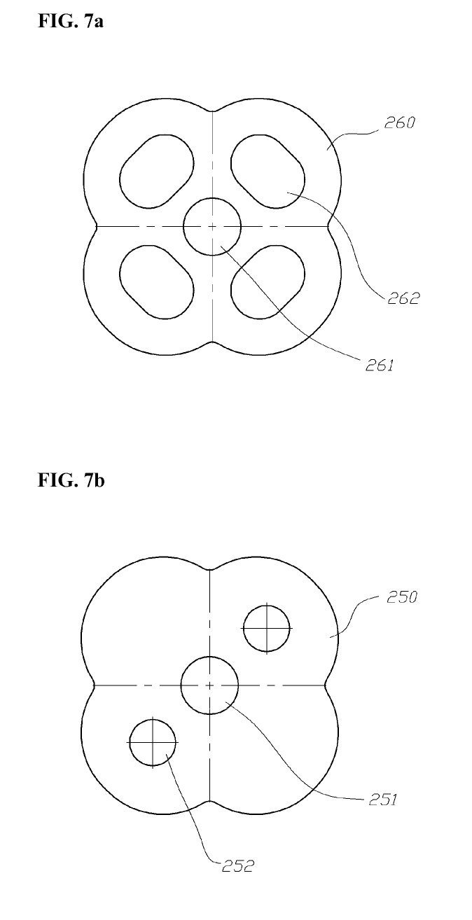

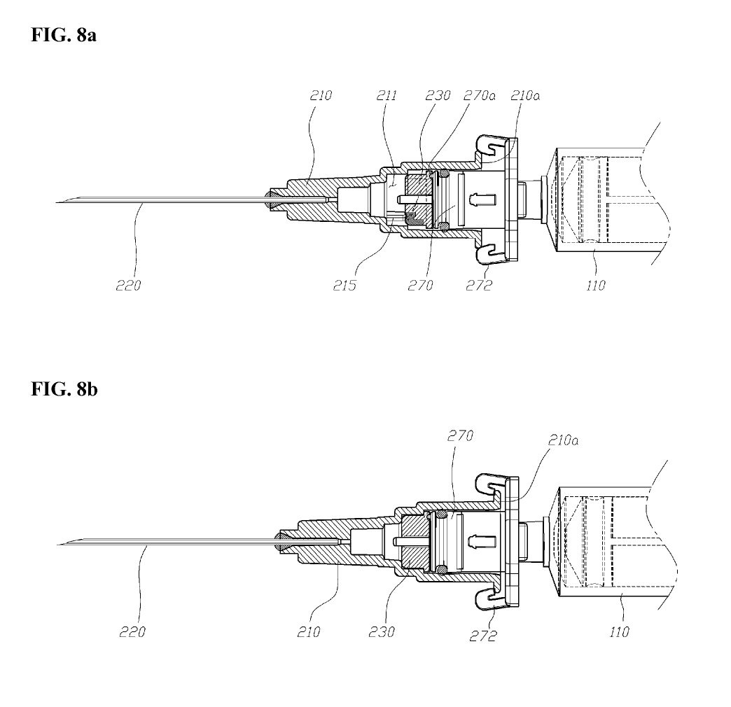

Moreover, FIGS. 4a and 4b are respectively a perspective view and a sectional view of a needle block applied to the syringe according to the preferred embodiment of the present invention, FIGS. 5a and 5b are respectively a perspective view and a sectional view of a filter block applied to the syringe according to the preferred embodiment of the present invention, FIG. 6 is an enlarged sectional view of the essential parts of the syringe, FIGS. 7a and 7b are front views of a filter and an adjustment member applied to the syringe according to the preferred embodiment of the present invention, and FIGS. 8a and 8b are sectional views showing an operation process of the syringe according to the preferred embodiment of the present invention.

Referring to the drawings, the syringe 100 according to the preferred embodiment of the present invention includes a needle holder 210, a filter block 230, a filter 250, an adjustment member 260, and a connecting cap 270 which are joined to the front of a cylinder 110 in order to filter foreign matters (pieces of broken glass of an ampoule, and others) contained in injection liquid.

The needle holder 210 is located at the frontmost portion of the syringe 100, and a needle (hereinafter, called ‘injection needle’) 220 is joined to the front portion of the needle holder 210 through a medium of sealant.

Moreover, the needle holder 210 is opened at the rear, and includes: a chamber 211 formed therein; first and second mounting parts 212 and 213 disposed at the rear side of the chamber 211 in a stepped form; and a pressure rib 214 formed at the front end of the first mounting part 212.

The needle holder 210 has at least one key hole 215 formed at one portion of the second mounting part 213. Here, the key hole 215 protrudes upward as shown in the drawings and a key 273 upwardly protruding from the filter block 230 is inserted into the key hole 215 so as to prevent vibration and rotation.

The filter block 230 is mounted inside the first mounting part 212 and rotates while the needle block 210 carries out a rectilinear motion so as to change a movement route of the injection liquid, such that the injection liquid does not go through the filter during inhalation of the injection liquid into the syringe but goes through the filter during injection of the injection liquid into the human body.

The filter block 230 includes a shaft hole 231 formed at the center and passing holes 232 formed around the shaft hole 231 so as to selectively communicate with an inhalation path 275 and a discharge path 276 formed in the connecting cap 270 which will be described later.

As shown in the drawings, the filter block 230 includes a spiral groove 234 formed in an outer circumferential surface for fitting the pressure rib 214 thereto and a stopper 234a formed at the lowest point of the spiral groove 234.

In the meantime, the spiral groove 234 is formed in the range of 90 degrees when the filter block 230 is viewed from the front (in the direction that the filter block 230 is seen in a circular shape), and two spiral grooves 234 may be formed at two places with a phase difference of 180 degrees.

Additionally, the filter block 230 further includes a concave groove 235 which is formed at an edge of a rear face thereof and to which a protrusion 270a formed at the front face of the connecting cap 270 is inserted. The concave groove 235 is formed at an angle of 90 degrees and serves to promote rotation of the filter block 230 and to guide the filter block to a correct rotation.

The reference numeral 236 designates a fixing protrusion for fixing a pad 260 which will be described later. That is, as shown in the drawings, the fixing protrusion 236 is formed in an inverted chevron shape in order to prick and fix the pad 260 like a needle, such that the pad 260 can be rotated at the same time when the filter block is rotated due to the route conversion.

The filter 250 is formed in a pad type and is inserted into a seating recess 279 formed in the front face of the connecting cap 270, and the pad 260 is located on the front face of the filter 250. As shown in the drawings, the filter 250 has a shaft hole 251 formed at the center and two holes 252 formed around the shaft hole 251, such that the filter 250 is assembled to the connecting cap 270 in a state where the two holes 252 are matched to the inhalation path when the filter 250 is inserted into the connecting cap 270.

Moreover, the pad 260 is molded of a silicon material and serves to enhance contactability between the filter block and the connecting cap, to prevent a leakage of the injection liquid to the outside, and to prevent air or foreign matters from flowing into the piston from the outside. The pad 260 has a shaft hole 261 formed at the center and four or two holes formed around the shaft hole 261.

The connecting cap 270 is located at the rearmost portion of the filter member 200, and its front portion is forcedly pressed to the second mounting part 213 of the needle holder 210 and its rear portion is opened in such a fashion that the neck part 112 of the cylinder 110 of the syringe 100 is forcedly pressed into the rear portion of the connecting cap 270.

The connecting cap 270 includes: a joining shaft 271 formed at the front face so as to pass through the shaft hole 231 of the filter block 230; and an inhalation path 275 and a discharge path 276 formed symmetrically around the joining shaft 271 of the front face so as to be selectively connected to the passing hole 232 formed in the filter block. Meanwhile, there are two inhalation paths and two discharge paths which are respectively formed in a symmetrical form, and an angle θ between the inhalation path 275 and the discharge path 276 is 90 degrees.

The protrusion 270a is formed at the front face of the connecting cap 270, the seating recess 279 to which the filter and the pad are inserted is formed inside the protrusion, and the key 273 is formed at the outer face of the connecting cap 270.

The connecting cap 270 further includes at least one annular protrusion 274 formed on the front portion of the outer circumference, namely, on the outer circumferential surface which is inserted into the second mounting part 213 of the needle holder 210, in such a way as to be forcedly pressed to the inner wall of the second mounting part.

Moreover, the connecting cap 270 has two hooks 272 formed at the rear face in a symmetrical form. The hooks 272 fix the needle holder 210, such that the user can inject the injection liquid stably and find that the syringe was used previously.

The reference numeral 277 designates a seal ring and 278 designates a recess to which the seal ring is inserted. Therefore, the syringe can keep sealability and a vacuum state when the connecting cap 270 is assembled to the needle holder.

In the meantime, the present invention is characterized in that the user can know whether or not the route is correctly changed when the route is changed for injecting the injection liquid. For this, the filter block 230 has a cognition member 233 disposed at the outer face. The cognition member 233 has various colors, such as red color or green color, and the needle holder 210 has a transparent window for allowing the user to check the color of the cognition member 233.

For instance, when the user pushes the needle holder to rotate the filter block at an angle of 90 degrees, because the cognition member 233 of the filter block is located on the transparent window, the user can find conversion of the route. On the other hand, in the case that the filter block is not rotated smoothly, the cognition member does not reach the transparent window, and it means that the route has not been converted properly.

Hereinafter, the operational process of the syringe according to the preferred embodiment of the present invention will be described.

The filter 250 and the pad 260 are inserted into the seating recess 279 of the connecting cap 270 and the joining shaft 271 of the connecting cap 270 is inserted into the shaft hole 231 of the filter block 230, such that the filter block 230 and the connecting cap 270 are joined to each other.

Next, the filter block 230 is inserted into the first mounting part 212 through the opened rear portion of the needle holder 210. Therefore, the connecting cap 270 joined with the filter block 230 is also pressed into the second mounting part 213. In the above-mentioned process, the key 273 of the connecting cap 270 is inserted and joined into the key hole 215 of the needle holder 210 so as to enhance accuracy in joining and to prevent an arbitrary rotation of the connecting cap 270.

Moreover, the protrusion 270a is fit into the concave groove 235 of the filter block 230 so as to promote the rotation, and in this instance, the pressure rib 214 of the needle holder 210 gets in contact with the highest point of the spiral groove 234 of the filter block 230, and the inhalation path 275 of the connecting cap 270 and the passing hole 232 are located to communicate with each other.

Next, the neck part 112 of the cylinder 110 is forcedly pressed and joined to the opened rear portion of the connecting cap 270, and then, the joining process is finished completely.

In the above state, when the user breaks the top of the ampoule in order to open the ampoule, inserts the needle 120 into the opened portion of the ampoule and retreats the piston, the injection liquid inside the ampoule is inhaled into the cylinder 110 of the syringe 100 through the needle holder 210, the passing hole 232 of the filter block 230 and the inhalation path 275 of the connecting cap 270.

In this instance, because there is no filtering action, pieces of broken glass formed when the ampoule is broken may be contained in the injection liquid and inhaled into the cylinder 110, but the syringe can easily inhale the injection liquid because the injection liquid does not go through the filter 250 when the injection liquid is inhaled into the syringe.

When the syringe completely inhales the injection liquid, the user pushes the needle holder 210 in the direction of the cylinder 110. Then, the pressure rib 214 slides along the spiral groove 234 of the filter block 230 so as to rotate the filter block at the angle of 90 degrees, and then, is stopped by the stopper 234a at the lowest point of the spiral groove 234.

Accordingly, the passing hole 232 of the filter block 230 communicates with the discharge path 276 of the connecting cap 270. In this instance, the user can find whether or not the filter block is located at the correct position through the cognition member 233 and the transparent window and can inject the injection liquid into the patient's body in a state where the needle holder 210 is perfectly fixed because the needle holder 210 is joined to the hook 272 disposed on the connecting cap 270. Additionally, the patient can visually judge whether or not the syringe is reused according to whether or not the needle holder 210 is joined to the hook.

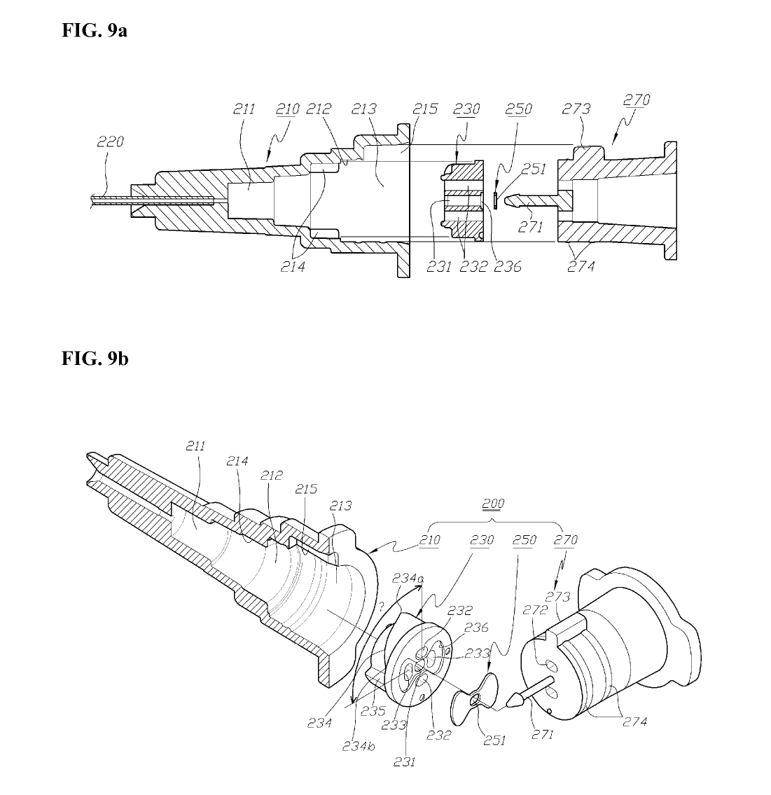

FIGS. 9a and 9b illustrate a syringe according to another preferred embodiment of the present invention.

Differently from the first preferred embodiment of the present invention, in this preferred embodiment, the filter block 230 includes: an inhalation path 232 and a discharge path 233; a shaft hole 231 formed at the center; a seating recess 236 formed in the rear face for allowing insertion of the filter 250; and two spiral grooves 234 and 235 symmetrically formed on the outer face.

The reference numerals 234a and 234b designates starting and ending portions of the spiral grooves, and the spiral grooves are formed in such a way as to be rotatable at the angle of 90 degrees.

The connecting cap 270 has a joining shaft 271 protrudingly formed at the center of the front face, and communication holes 272 formed around the joining shaft 271 for selectively communicating with the inhalation path and the discharge path. Of course, the connecting cap 270 has a key 273 and an annular protrusion 274 formed on the outer face.

The needle holder 210 according to the second preferred embodiment is the same as the needle holder according to the first preferred embodiment. In other words, the needle holder 210 is opened at the rear portion, and includes: a chamber 211 formed therein; first and second mounting parts 212 and 213 disposed at the rear side of the chamber 211 in a stepped form; and a pressure rib 214 formed at the front end of the first mounting part 212.

The needle holder 210 has at least one key hole 215 formed at one portion of the second mounting part 213. Here, as shown in the drawings, the key hole 215 protrudes upward as shown in the drawings and a key 273 upwardly protruding from the filter block 230 is inserted into the key hole 215 so as to prevent vibration and rotation.

The reference numeral 251 designates a shaft hole. In this embodiment, the filter block 230 has a seating recess 236 formed at the discharge path 233 of the filter block 230 for seating the filter 250 in the seating recess 236, such that the filter 250 is selectively communicated with the through holes 272 formed in the connecting cap 270 while the filter block 230 rotates, and thus, the syringe carries out the filtering action just when the injection liquid is injected into the patient's body. Furthermore, in this embodiment, the syringe does not include a hook and a pad so as to reduce manufacturing costs.

While the present invention has been described with reference to the particular illustrative embodiments, it is not to be restricted by the exemplary embodiments and can be changed or modified into various forms without departing from the scope of the present invention. Moreover, it will be understood by those of ordinary skill in the art that various changes, modifications and equivalents may be made therein without changing the essential characteristics and scope of the present invention within the spirit and scope of the invention defined by the claims.

What is claimed is:

1. A syringe comprising:
a cylinder having a piston;
a needle holder having an injection needle disposed at the front end thereof;
a filter block fit into the needle holder, the filter block having a through hole formed therein;
a connecting cap disposed at a rear portion of the needle holder and having an inhalation path and a discharge path, the connecting cap having a front end fit into the rear portion of the needle holder in a state where a front face of the connecting cap is in contact with the filter block and a rear end connected to the cylinder; and
wherein injection liquid is inhaled into the cylinder through the through hole and the inhalation path, and after inhalation of the injection liquid, the filter block is rotated so as to communicate the discharge path with the through hole, such that the inhaled injection liquid is injected into a patient's body while passing through a filter disposed on the discharge path.

2. The syringe according to claim 1, wherein the spiral groove formed on the outer face of the filter block allows the filter block to rotate at an angle of 90 degrees.

3. A syringe for inhaling and discharging injection liquid by a reciprocating motion of a piston of a cylinder, comprising:
a connecting cap joined to the front end of the cylinder, the connecting cap having an inhalation path and a discharge path communicating with the inside of the cylinder;
a filter block assembled to the front face of the connecting cap, the filter block having a passing hole selectively communicating with the inhalation path and the discharge path and a spiral groove formed on the outer face of the filter block;
a filter fit to the front face of the connecting cap, the filter having a hole formed at the same position as the inhalation path so as to pass the inhaled injection liquid without filtering;
a pad located on the front face of the filter and having a hole communicating with the passing hole of the filter block;
a needle holder having an injection needle disposed at the front portion and first and second mounting parts disposed therein, the filter block and the connecting cap being fit to the needle holder in order, the needle holder having a pressure rib disposed in the first mounting part inserted into the spiral groove; and
wherein the injection liquid is inhaled into the cylinder through the injection needle, the passing hole and the inhalation path in order, and is injected into a patient's body through the discharge path, the filter and the passing hole in order while the filter holder is rotated at the same time with a movement of the pressure rib along the spiral groove when the needle holder carries out a rectilinear motion.

4. The syringe according to claim 2, wherein the spiral groove formed on the outer face of the filter block allows the filter block to rotate at an angle of 90 degrees.

5. The syringe according to claim 2, wherein the filter block and the filter respectively comprise shaft holes formed at the center thereof, a joining shaft protrudingly formed at the center of the connecting cap passes through the shaft holes, and a key protrudingly formed on the outer face of the filter block is forcedly fit to a key hole formed in the inner face of the needle holder.

6. The syringe according to claim 2, wherein the connecting cap comprises hooks disposed symmetrically on the rear face thereof and the needle holder is joined and fixed to the hooks.

7. The syringe according to claim 2, wherein the filter block comprises a cognition member having various colors, and the needle holder comprises a transparent window for allowing a user to see the cognition member therethrough after rotation of the filter block.

8. The syringe according to claim 7, wherein the cognition member is assembled to the outer face of the filter block by a forced fit.

9. The syringe according to claim 2, wherein the connecting cap comprises a protrusion formed on the front face and the filter block comprises a concave groove formed in the rear face to which the protrusion is inserted, and the concave groove has a rotation radius of 90 degrees.

10. A syringe for inhaling and discharging injection liquid by a reciprocating motion of a piston of a cylinder, comprising:
a connecting cap joined to the front end of the cylinder, the connecting cap having a through hole communicating with the inside of the cylinder;
a filter block assembled to the front face of the connecting cap, the filter block having an inhalation path and a discharge path which are selectively communicated with the through hole and a spiral groove formed on the outer face of the filter block;
a filter fit to the rear face of the filter block, the filter having a hole formed at the same position as the inhalation path so as to pass the inhaled injection liquid without filtering;
a needle holder having an injection needle disposed at the front portion and first and second mounting parts disposed therein, the filter block and the connecting cap being fit to the needle holder in order, the needle holder having a pressure rib disposed in the first mounting part inserted into the spiral groove; and
wherein the injection liquid is inhaled into the cylinder through the injection needle, the passing hole and the inhalation path in order, and is injected into a patient's body through the discharge path, the filter and the passing hole in order while the filter holder is rotated at the same time with a movement of the pressure rib along the spiral groove when the needle holder carries out a rectilinear motion.

11. The syringe according to claim 10, wherein the spiral groove formed on the outer face of the filter block allows the filter block to rotate at an angle of 90 degrees.

12. The syringe according to claim 10, wherein the filter block and the filter respectively comprise shaft holes formed at the center thereof, a joining shaft protrudingly formed at the center of the connecting cap passes through the shaft holes, and a key protrudingly formed on the outer face of the filter block is forcedly fit to a key hole formed in the inner face of the needle holder.
